(19) United States
(12) Patent Application Publication
(10) Pub. No.: US 2020/0277632 A1
(43) Pub. Date: Sep. 3, 2020

(54) GLUCOAMYLASE AND METHODS OF USE, THEREOF

(71) Applicant: DANISCO US INC., Palo Alto, CA (US)

(72) Inventors: Zhongmei Tang, Palo Alto, CA (US); Helong Hao, Shanghai (CN); Zhiyong Xie, Shanghai (CN); Zhenghong Zhang, Palo Alto, CA (US)

(21) Appl. No.: 16/645,687

(22) PCT Filed: Aug. 30, 2018
(86) PCT No.: PCT/US2018/048710

§ 371 Date: Mar. 9, 2020

(30) Foreign Application Priority Data
Sep. 11, 2017 (CN) PCT/CN2017/101177

(51) Int. Cl. C12P 7/06; C13K 1/06; C12N 9/34

(52) U.S. Cl. CPC C12P 7/06; C12Y 302/01003; C12N 9/2428; C13K 1/06

(57) ABSTRACT

Described are methods of saccharifying starch-containing materials using a glucoamylase, as well as the methods of producing fermentation products and the fermentation products produced by the method thereof.

### FIELD OF THE INVENTION

The present disclosure relates to methods of saccharifying starch-containing materials using a glucoamylase. Moreover, the disclosure relates to methods of producing fermentation products as well as the fermentation products produced by the method thereof.

### BACKGROUND

Glucoamylase (1,4-alpha-D-glucan glucohydrolase, EC 3.2.1.3) is an enzyme, which catalyzes the release of D-glucose from the non-reducing ends of starch or related oligo- and poly-saccharide molecules. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase, since they secrete large quantities of the enzyme extracellularly. However, the commercially used fungal glucoamylases have certain limitations such as slow catalytic activity that increase the process cost.

There continues to be a need to search for new glucoamylases to improve the efficiency of saccharification and provide a high yield in fermentation products, such as ethanol production, including one-step ethanol fermentation processes from un-gelatinized raw (or uncooked) starch.

### SUMMARY

The present disclosure relates to the methods of saccharifying starch-containing materials using or applying the polypeptides or compositions. Aspects and embodiments of the methods are described in the following, independently-numbered paragraphs.
1. In one aspect, a method for saccharifying a starch substrate, comprising contacting the starch substrate with a glucoamylase selected from the group consisting of:
   a) a polypeptide having the amino acid sequence of SEQ ID NO: 3;
   b) a polypeptide having at least 81% identity to the amino acid sequence of SEQ ID NO: 3;
   c) a polypeptide having at least 82% identity to a catalytic domain of SEQ ID NO: 3; or
   d) a polypeptide having at least 82% identity to a linker and a catalytic domain of SEQ ID NO: 3.
2. In some embodiments of the method of paragraph 1, wherein saccharifying the starch substrate results in a high glucose syrup.
3. In some embodiments of the method of paragraph 1 or 2, wherein the high glucose syrup comprises an amount of glucose selected from the list consisting of at least 95.5% glucose, at least 95.6% glucose, at least 95.7% glucose, at least 95.8% glucose, at least 95.9% glucose, at least 96% glucose, at least 96.1% glucose, at least 96.2% glucose, at least 96.3% glucose, at least 96.4% glucose, at least 96.5% glucose and at least 97% glucose.
4. In some embodiments of the method of any one of paragraphs 1-3, further comprising fermenting the high glucose syrup to an end product.
5. In some embodiments of the method of paragraph 4, wherein saccharifying and fermenting are carried out as a simultaneous saccharification and fermentation (SSF) process.
6. In some embodiments of the method of paragraph 4 or 5, wherein the end product is alcohol, for example, ethanol.
7. In some embodiments of the method of paragraph 4 or 5, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.
8. In some embodiments of the method of any one of paragraphs 1-7, wherein the starch substrate is about, 5% to 99%, 15% to 50% or 40-99% dry solid (DS).
9. In some embodiments of the method of any one of paragraphs 1-8, wherein the starch substrate is selected from wheat, barley, corn, rye, rice, sorghum, bran, cassava, milo, millet, potato, sweet potato, tapioca, and any combination thereof.
10. In some embodiments of the method of any one of paragraphs 1-9, wherein the starch substrate comprises liquefied starch, gelatinized starch, or granular starch.
11. In some embodiments of the method of any one of paragraphs 1-10, further comprising adding a hexokinase, a xylanase, a glucose isomerase, a xylose isomerase, a phosphatase, a phytase, a pullulanase, a beta-amylase, an a-amylase, a protease, a cellulase, a hemicellulase, a lipase, a cutinase, a trehalase, an isoamylase, a redox enzyme, an esterase, a transferase, a pectinase, a hydrolase, an alpha-glucosidase, a beta-glucosidase, or a combination thereof to the starch substrate.
12. In another aspect, a method for saccharifying and fermenting a starch substrate to produce an end product, comprising contacting the starch substrate with a glucoamylase selected from the group consisting of:
   a) a polypeptide having the amino acid sequence of SEQ ID NO: 3;
   b) a polypeptide having at least 81% identity to the amino acid sequence of SEQ ID NO: 3;
   c) a polypeptide having at least 82% identity to a catalytic domain of SEQ ID NO: 3; or
   d) a polypeptide having at least 82% identity to a linker and a catalytic domain of SEQ ID NO: 3.
13. In some embodiments of the method of paragraph 12, wherein saccharifying and fermenting are carried out as a simultaneous saccharification and fermentation (SSF) process.
14. In some embodiments of the method of paragraph 12 or 13, wherein the end product is alcohol, for example, ethanol.
15. In some embodiments of the method of paragraph 12, wherein the saccharified starch substrate results in a reduced level of DP3+ and an increased level of DP1 compared to contacting the same starch substrate with AnGA.
16. In some embodiments of the method of paragraph 12 or 13, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.
17. In another aspect, a method of producing a fermented beverage, wherein the method comprises the step of contacting a mash and/or a wort with a glucoamylase selected from the group consisting of:
   a) a polypeptide having the amino acid sequence of SEQ ID NO: 3;
   b) a polypeptide having at least 81% identity to the amino acid sequence of SEQ ID NO: 3;
   c) a polypeptide having at least 82% identity to a catalytic domain of SEQ ID NO: 3; or
   d) a polypeptide having at least 82% identity to a linker and a catalytic domain of SEQ ID NO: 3.

### BRIEF DESCRIPTION OF THE DRAWINGS

FIG. 1 is the map of pGX256-PspGA3

### DETAILED DESCRIPTION

The present disclosure relates to methods of saccharifying starch-containing materials using. Moreover, the disclosure relates to methods of producing fermentation products as well as the fermentation products produced by the method thereof.

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

### I. Definition

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules.

The polypeptides of the present invention have at least 81%, even more preferably at least 90%, most preferably at least 95%, and even most preferably at least 100% of the glucoamylase activity of the polypeptide of SEQ ID NO: 3.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc. In one aspect, the mature polypeptide is amino acids 28 to 586 of SEQ ID NO: 2 based on the analysis of MALDI-ISD (Alphalyse, Inc.), and amino acids 1 to 27 of SEQ ID NO: 2 are a signal peptide.

The term “nucleic acid” encompasses DNA, RNA, heteroduplexes, and synthetic molecules capable of encoding a polypeptide. Nucleic acids may be single stranded or double stranded, and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

A “synthetic” molecule is produced by in vitro chemical or enzymatic synthesis rather than by an organism.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | TUB |
| Delay divergent sequences %: | 40 |
| Gap separation distance: | 8 |
| DNA transitions weight: | 0.50 |
| List hydrophilic residues: | GP SNDQEKR |
| Use negative matrix: | OFF |
| Toggle Residue specific penalties: | ON |
| Toggle hydrophilic penalties: | ON |
| Toggle end gap separation penalty | OFF. |

The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with the glucoamylase of SEQ ID NO: 2 or the mature polypeptide thereof.

The term “polypeptide fragment” is defined herein as a polypeptide having one or more (e.g., several) amino acids deleted from the amino and/or carboxyl terminus of the mature polypeptide of SEQ ID NO: 2; or a homologous sequence thereof; wherein the fragment has glucoamylase activity.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (ti/2) given in minutes, hours, or days, during which half the enzyme activity is lost under defined conditions. The half-life may be calculated by measuring residual alpha-amylase activity for example following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

“Dry solid content” refers to the percentage of dry solids both dissolved and dispersed as a percentage by weight with respect to the water in which the dry solids are dispersed and/or dissolved. The initial dry solid content of starch is the weight of granular starch corrected for moisture content over the weight of granular starch plus weight of water. Subsequent dry solid content can be determined from the initial content adjusted for any water added or lost and for chemical gain. Subsequent dissolved dry solid content can be measured from refractive index as indicated below. 8

The term “high DS” refers to aqueous starch slurry with a dry solid content greater than 38% (wt/wt).

“Dry substance starch” refers to the dry starch content of a substrate, such as a starch slurry, and can be determined by subtracting from the mass of the substrate any contribution of non-starch components such as protein, fiber, and water. For example, if a granular starch slurry has a water content of 20% (wt/wt)., and a protein content of 1% (wt/wt), then 100 kg of granular starch has a dry starch content of 79 kg. Dry substance starch can be used in determining how many units of enzymes to use.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+ (>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate. Those skilled in the art will recognize that mixing solutions can bring about “contacting.” An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “pullulanase” also called debranching enzyme (E.C. 3.2.1.41, pullulan 6-glucanohydrolase), is capable of hydrolyzing alpha 1-6 glucosidic linkages in an amylopectin molecule.

The term “about” refers to ±15% to the referenced value.

The term “comprising” and its cognates are used in their inclusive sense; that is, equivalent to the term “including” and its corresponding cognates.

The following abbreviations/acronyms have the following meanings unless otherwise specified:

EC enzyme commission

CAZy carbohydrate active enzyme

w/v weight/volume

w/w weight/weight

v/v volume/volume

wt % weight percent

° C. degrees Centigrade

g or gm gram

microgram

mg milligram

kg kilogram

μL and μl microliter

mL and ml milliliter

mm millimeter

μm micrometer

mol mole

mmol millimole

M molar

mM millimolar

micromolar

nm nanometer

U unit

ppm parts per million

hr and h hour

EtOH ethanol

### II. Polypeptides Having Glucoamylase Activity Used in this Invention

In a first aspect, the present invention relates to polypeptides comprising an amino acid sequence having preferably at least 81%, at least 83%, at least 85%, at least 90%, at least 92%, at least 93%, at least 94%, at least 95%, at least 96%, at least 97%, at least 98%, and even at least 99%, amino acid sequence identity to the polypeptide of SEQ ID NO: 3, and having glucoamylase activity.

In some embodiments, the polypeptides of the present invention are the homologous polypeptides comprising amino acid sequences differ by no more than ten amino acids, no more than nine amino acids, no more than eight amino acids, no more than seven amino acids, no more than six amino acids no more than five amino acids, no more than four amino acids, no more than three amino acids, no more than two amino acids, and even no more than one amino acid from the polypeptide of SEQ ID NO: 3.

In some embodiments, the polypeptides of the present invention are the variants of polypeptide of SEQ ID NO: 3, or a fragment thereof having glucoamylase activity.

In some embodiments, the polypeptides of the present invention are the catalytic regions comprising the amino acids 28 to 478 of SEQ ID NO: 2 predicted by Clustalx https://www.ncbi.nlm.nih.gov/pubmed/17846036.

In some embodiments, the polypeptides of the present invention are the catalytic regions and linker regions comprising the amino acids 28 to 485 of SEQ ID NO: 2 predicted by Clustalx https://www.ncbi.nlm.nih.gov/pubmed/17846036.

In some embodiments, the polypeptides of the present invention have the pullulan-hydrolyzing activity.

In a second aspect, the present glucoamylases comprise conservative substitution of one or several amino acid residues relative to the amino acid sequence of SEQ ID NO: 3. Exemplary conservative amino acid substitutions are listed in the Table 1. Some conservative mutations can be produced by genetic manipulation, while others are produced by introducing synthetic amino acids into a polypeptide by other means.

TABLE 1
Conservative amino acid substitutions

| For Amino | | |
|---|---|---|
| Acid | Code | Replace with any of |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, the present glucoamylase comprises a deletion, substitution, insertion, or addition of one or a few amino acid residues relative to the amino acid sequence of SEQ ID NO: 3 or a homologous sequence thereof. In some embodiments, the present glucoamylases are derived from the amino acid sequence of SEQ ID NO: 3 by conservative substitution of one or several amino acid residues. In all cases, the expression “one or a few amino acid residues” refers to 10 or less, i.e., 1, 2, 3, 4, 5, 6, 7, 8, 9, or 10, amino acid residues. The amino acid substitutions, deletions and/or insertions of the mature polypeptide of SEQ ID NO: 3 can be at most 10, at most 9, more at most 8, more at most 7, more at most 6, more at most 5, more at most 4, even more at most 3, at most 2, and even at most 1.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The glucoamylase may be a “chimeric” or “hybrid” polypeptide, in that it includes at least a portion from a first glucoamylase, and at least a portion from a second amylase, glucoamylase, beta-amylase, alpha-glucosidase or other starch degrading enzymes, or even other glycosyl hydrolases, such as, without limitation, cellulases, hemicellulases, etc. (including such chimeric amylases that have recently been “rediscovered” as domain-swap amylases). The present glucoamylases may further include heterologous signal sequence, an epitope to allow tracking or purification, or the like.

### III. Production of Glucoamylase

The present glucoamylases can be produced in host cells, for example, by secretion or intracellular expression. A cultured cell material (e.g., a whole-cell broth) comprising a glucoamylase can be obtained following secretion of the glucoamylase into the cell medium. Optionally, the glucoamylase can be isolated from the host cells, or even isolated from the cell broth, depending on the desired purity of the final glucoamylase. A gene encoding a glucoamylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae, Trichoderma reesi or Myceliopthora thermophila. Other host cells include bacterial cells, e.g., Bacillus subtilis or B. licheniformis, as well as Streptomyces.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

### 3.1. Vectors

A DNA construct comprising a nucleic acid encoding a glucoamylase polypeptide can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding a glucoamylase polypeptide of the present disclosure can be incorporated into a vector. Vectors can be transferred to a host cell using known transformation techniques, such as those disclosed below.

A suitable vector may be one that can be transformed into and replicated within a host cell. For example, a vector comprising a nucleic acid encoding a glucoamylase polypeptide of the present disclosure can be transformed and replicated in a bacterial host cell as a means of propagating and amplifying the vector. The vector may also be suitably transformed into an expression host, such that the encoding polynucleotide is expressed as a functional glucoamylase enzyme.

A representative useful vector is pTrex3gM (see, Published US Patent Application 20130323798) and pTTT (see, Published US Patent Application 20110020899), which can be inserted into genome of host. The vectors pTrex3gM and pTTT can both be modified with routine skill such that they comprise and express a polynucleotide encoding a glucoamylase polypeptide of the invention.

A vector useful for this purpose typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the glucoamylase to a host cell organelle such as a peroxisome, or to a particular host cell compartment. Such a targeting sequence includes but is not limited to the sequence, SKL. For expression under the direction of control sequences, the nucleic acid sequence of the glucoamylase is operably linked to the control sequences in proper manner with respect to expression.

A polynucleotide encoding a glucoamylase polypeptide of the present invention can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding a glucoamylase, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a glucoamylase is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40(2): 158-65.

The coding sequence can be operably linked to a signal sequence. The DNA encoding the signal sequence may be a DNA sequence naturally associated with the glucoamylase gene of interest to be expressed, or may be from a different genus or species as the glucoamylase. A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding a glucoamylase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

Intracellular expression may be advantageous in some respects, e.g., when using certain bacteria or fungi as host cells to produce large amounts of alpha-glucosidase for subsequent enrichment or purification. Alternatively, extracellular secretion of glucoamylase into the culture medium can also be used to make a cultured cell material comprising the isolated glucoamylase.

### 3.2. Transformation and Culture of Host Cells

An isolated cell, either comprising a DNA construct or an expression vector, is advantageously used as a host cell in the recombinant production of a glucoamylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A glucoamylase expressed by a fungal host cell can be glycosylated, i.e., will comprise a glycosyl moiety. The glycosylation pattern can be the same or different as present in the wild-type glucoamylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding an alpha-glucosidase is stably integrated into a host cell chromosome. Transformants are then selected and purified by known techniques.

### 3.3. Expression and Fermentation

A method of producing a glucoamylase may comprise cultivating a host cell under conditions conducive to the production of the enzyme and recovering the enzyme from the cells and/or culture medium.

The medium used to cultivate the cells may be any conventional medium suitable for growing the host cell and obtaining expression of a glucoamylase polypeptide. Suitable media and media components are available from commercial suppliers or may be prepared according to published recipes (e.g., as described in catalogues of the American Type Culture Collection).

Any of the fermentation methods well known in the art can suitably used to ferment the transformed or the derivative fungal strain as described above. In some embodiments, fungal cells are grown under batch or continuous fermentation conditions.

### 3.4. Methods for Enriching and Purification

Separation and concentration techniques are known in the art and conventional methods can be used to prepare a concentrated solution or broth comprising a glucoamylase polypeptide of the invention.

After fermentation, a fermentation broth is obtained, the microbial cells and various suspended solids, including residual raw fermentation materials, are removed by conventional separation techniques in order to obtain a glucoamylase solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth comprising a glucoamylase polypeptide to optimize recovery. Use of un-concentrated solutions or broth would typically increase incubation time in order to collect the enriched or purified enzyme precipitate.

### IV. Compositions

The present invention also relates to compositions comprising a polypeptide of the present invention. In some embodiments, a polypeptide comprising an amino acid sequence that is at least about 70%, at least about 75%, at least about 80%, at least about 85%, at least about 90%, at least about 95%, identical to that of SEQ ID NO: # can also be used in the enzyme composition. Preferably, the compositions are formulated to provide desirable characteristics such as low color, low odor and acceptable storage stability.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present glucoamylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc, for example, endogenous plant (including algal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a cream or in dry form along with suitable stabilizers. Such cells may further express additional polypeptides, such as those mentioned, above.

Examples are given below of preferred uses of the polypeptides or compositions of the invention. The dosage of the polypeptide composition of the invention and other conditions under which the composition is used may be determined on the basis of methods known in the art.

Above composition is suitable for use in liquefaction, saccharification, and/or fermentation process, preferably in starch conversion, especially for producing syrup and fermentation products, such as ethanol.

### V. Use

The present invention is also directed to use of a polypeptide or composition of the present invention in a liquefaction, a saccharification and/or a fermentation process. The polypeptide or composition may be used in a single process, for example, in a liquefaction process, a saccharification process, or a fermentation process. The polypeptide or composition may also be used in a combination of processes for example in a liquefaction and saccharification process, in a liquefaction and fermentation process, or in a saccharification and fermentation process, preferably in relation to starch conversion.

### 5.1 Saccharification

The liquefied starch may be saccharified into a syrup rich in lower DP (e.g., DP1+DP2) saccharides, using alpha-amylases and glucoamylases, optionally in the presence of another enzyme(s). The exact composition of the products of saccharification depends on the combination of enzymes used, as well as the type of starch processed. Advantageously, the syrup obtainable using the provided glucoamylases may contain a weight percent of DP2 of the total oligosaccharides in the saccharified starch exceeding 30%, e.g., 45%-65% or 55%-65%. The weight percent of (DP1+DP2) in the saccharified starch may exceed about 70%, e.g., 75%-85% or 80%-85%.

Whereas liquefaction is generally run as a continuous process, saccharification is often conducted as a batch process. Saccharification conditions are dependent upon the nature of the liquefact and type of enzymes available. In some cases, a saccharification process may involve temperatures of about 60-65° C. and a pH of about 4.0-4.5, e.g., pH 4.3. Saccharification may be performed, for example, at a temperature between about 40° C., about 50° C., or about 55° C. to about 60° C. or about 65° C., necessitating cooling of the Liquefact. The pH may also be adjusted as needed. Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours. However, it is common only to do a pre-saccharification of typically 40-90 minutes at a temperature between 30-65° C., typically about 60° C., followed by complete saccharification in a simultaneous saccharification and fermentation (SSF). In one embodiment a process of the invention includes pre-saccharifying starch-containing material before simultaneous saccharification and fermentation (SSF) process. The pre-saccharification can be carried out at a high temperature (for example, 50-85° C., preferably 60-75° C.) before moving into SSF. Preferredly, saccharification optimally is conducted at a higher temperature range of about 30° C. to about 75° C., e.g., 45° C.-75° C. or 50° C.-75° C. By conducting the sacchanfication processes at higher temperatures, the process can be carried out in a shorter period of time or alternatively the process can be carried out using lower enzyme dosage. Furthermore, the risk of microbial contamination is reduced when carrying the liquefaction and/or sacchanfication process at higher temperature.

In a preferred aspect of the present invention, the liquefaction and/or saccharification includes sequentially or simultaneously performed liquefaction and saccharification processes.

### 5.2 Fermentation

The soluble starch hydrolysate, particularly a glucose rich syrup, can be fermented by contacting the starch hydrolysate with a fermenting organism typically at a temperature around 32° C., such as from 30° C. to 35° C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas moblis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27:1049-56. Commercially available yeast includes, e.g., Red Star™/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties). The temperature and pH of the fermentation will depend upon the fermenting organism. Microorganisms that produce other metabolites, such as citric acid and lactic acid, by fermentation are also known in the art. See, e.g., Papagianni (2007) Biotechnol. Adv. 25:244-63; John et al. (2009) Biotechnol. Adv. 27:145-52.

The saccharification and fermentation processes may be carried out as an SSF process. An SSF process can be conducted with fungal cells that express and secrete glucoamylase continuously throughout SSF. The fungal cells expressing glucoamylase also can be the fermenting microorganism, e.g., an ethanologenic microorganism. Ethanol production thus can be carried out using a fungal cell that expresses sufficient glucoamylase so that less or no enzyme has to be added exogenously. The fungal host cell can be from an appropriately engineered fungal strain. Fungal host cells that express and secrete other enzymes, in addition to glucoamylase, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

### 5.3 Raw Starch Hydrolysis

The present invention provides a use of the glucoamylase of the invention for producing glucoses and the like from raw starch or granular starch. Generally, glucoamylase of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least an glucoamylase and/or amylase. Starch heated in water begins to gelatinize between 50° C. and 75° C., the exact temperature of gelatinization depends on the specific starch. For example, the gelatinization temperature may vary according to the plant species, to the particular variety of the plant species as well as with the growth conditions. In the context of this invention the gelatinization temperature of a given starch is the temperature at which birefringence is lost in 5% of the starch granules using the method described by Gorinstein. S. and Lii. C., Starch/Starke, Vol. 44 (12) pp. 461-466 (1992).

The glucoamylase of the invention may also be used in combination with an enzyme that hydrolyzes only alpha-(1,6)-glucosidic bonds in molecules comprising at least four glucosyl residues. Preferably, the glucoamylase of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

### 5.4 Fermentation Products

The term “fermentation product” means a product produced by a process including a fermentation process using a fermenting organism. Fermentation products contemplated according to the invention include alcohols (e.g., arabinitol, butanol, ethanol, glycerol, methanol, ethylene glycol, propylene glycol, butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glutaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H₂), carbon dioxide (CO₂), and carbon monoxide (CO)); antibiotics (e.g., penicillin and tetracycline); enzymes; vitamins (e.g., riboflavin, B₁₂, beta-carotene); and hormones.

In a preferred aspect the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, high-alcohol beer, low-alcohol beer, low-calorie beer or light beer. Preferred fermentation processes used include alcohol fermentation processes, which are well known in the art. Preferred fermentation processes are anaerobic fermentation processes, which are well known in the art.

### 5.5 Brewing

Processes for making beer are well known in the art. See, e.g., Wolfgang Kunze (2004) “Technology Brewing and Malting,” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer.

The brewing composition comprising a glucoamylase, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1

### Sequence of Paraphaeosphaeria sporulosa Glucoamylase (PspGA3)

The nucleic acid sequence for the PspGA3 gene (NCBI Reference No. KV441563.1), and the amino acid sequence of the predicted glucoamylase (NCBI Accession No. OAF98892.1) encoded by the PspGA3 gene were obtained in the NCBI Databases. The gene encoding PspGA3 is set forth as SEQ ID NO:1:

atgctcttcacctcagtcattcgggccdtccggcatccttgctttttacag
gcgctcatggcacgcctctcgagcgtcgtcagacttccgtcgacacgttcg
taacaagccagagatcggtttcgatccaaggtgtgctttctaacatcgggg
ctgatggctccaaggcgcaaggtgcagcagcgggcatcgtcgtggccagtc
cgtccaagtcaaacccagattgtaagtagaacaaactcaggcgcacattac
atgggttgacaagccatagactggtacacttggacaagagactctgctttg
acgtacaaagccctcgtcgagcgtttcatcggtggggacaccacactgaga
aagaagctcgatgagtacgtctcatcccaggcgtatctccagaccgtgtcg
aacccgtccggcggtcctgacaccggaggactcggcgagcccaagttcaac
gtcgaccgtactgcatttactggagcttggggtcgtcctcagcgcgatgga
ccacctctacgagccactgccttgacactctacgctaattggcttattgcc
aacggcaacaccacacaagctgcaaataccctttggcctgttattgccaag
gatcttgcctacgccgtcaagtactggaaccagaccggcttcgacttgtgg
gaagaggtcaatggctcatccttctttaccctcgcagctactcatcgtgcc
ctcgtagagggcgctgcactcgctactaagctctcaaagacatgtgacggg
tgtgctgccgcggccccacagatcctctgcttccttcaaagcttctggact
ggcagttacatcgattccaacatcaatgtgaatgatggtcgtactggcaaa
gacgtcaactccatcatctcatcgatccataccttcgacccagcgtcagcc
tgtaccgacgctactttccaaccttgctcttctcgcgctctgctcaaccac
aaggctgttactgactccttccgcaccgtgtacggcatcaacaagggtatc
gcccaaggacaagctgtcgccgttggaagatattctgaggatgtatactac
aatggcaacccgtggtatcttgcgacgctcgcggccgcagaacaactttac
gccgctgtctaccaatggaacaccataggctccatcaccgttgactctgta
tctcttccattcttcaaggacctcctgcccagcatcgccactggtacctac
gcctcgagctcggctaccttcgcatcgattgtctctgctgtcaaaacatac
ggtgacggctacgttgccgtggtccagaagtacactcctgccaatggcggg
ttggccgaacagttcgacaagagcagtggttccccactctctgccgttgac
ctgacttggtcttatgcggctttcctgaccgccaccgatcgtcgttctggg
tctgtgggtccttcttggggagagaagtccaacaacgttcctccgacatca
tgcacagctcctccttcctgcaatgtgcaagtcactttcaacgaacgcgtg
accaccgcctacggagacaacatcttcattgttggacagctcactcagctc
ggcaactgggatccgaacagcgctgttgcgctcagtgccagcaagtacacc
agcagtgacccgctctggtatgcaactgtcagcctccctgcctcaacttct
ttcgcatacaaatacatcaagaagacgtcgagcggcactgtcgtatgggag
agcgacccgaacaggagctacacaactgcaaccacttgtggaagcactgcg
actcagaacgacacatggagataa

The amino acid sequence of the PspGA3 precursor protein is set forth as SEQ ID NO: 2. The native signal peptide is shown in italics and underline.

MLFTSVIRALPASLLFTGAHGTPLERRQTSVDTFVTSQRSVSIQGVLSNIG
ADGSKAQGAAAGIVVASPSKSNPDYWYTWTRDSALTYKALVERFIGGDTTL
RKKLDEYVSSQAYLQTVSNPSGGPDTGGLGEPKFNVDRTAFTGAWGRPQRD
GPPLRATALTLYANWLIANGNTTQAANTLWPVIAKDLAYAVKYWNQTGFDL
WEEVNGSSFFTLAATHRALVEGAALATKLSKTCDGCAAAAPQILCFLQSFW
TGSYIDSNINVNDGRTGKDVNSIISSIHTFDPASACTDATFQPCSSRALLN
HKAVTDSFRTVYGINKGIAQGQAVAVGRYSEDVYYNGNPWYLATLAAAEQL
YAAVYQWNTIGSITVDSVSLPFFKDLLPSIATGTYASSSATFASIVSAVKT
YGDGYVAVVQKYTPANGGLAEQFDKSSGSPLSAVDLTWSYAAFLTATDRRS
GSVGPSWGEKSNNVPPTSCTAPPSCNVQVTFNERVTTAYGDNIFIVGQLTQ
LGNWDPNSAVALSASKYTSSDPLWYATVSLPASTSFAYKYIKKTSSGTVVW
ESDPNRSYTTATTCGSTATQNDTWR

The amino acid sequence of the mature form of PspGA3 confirmed by the analysis of MALDI-ISD (Alphalyse, Inc.) is set forth as SEQ ID NO:3:

QTSVDTFVTSQRSVSIQGVLSNIGADGSKAQGAAAGIVVASPSKSNPDYWY
TWTRDSALTYKALVERFIGGDTTLRKKLDEYVSSQAYLQTVSNPSGGPDTG
GLGEPKFNVDRTAFTGAWGRPQRDGPPLRATALTLYANWLIANGNTTQAAN
TLWPVIAKDLAYAVKYWNQTGFDLWEEVNGSSFFTLAATHRALVEGAALAT
KLSKTCDGCAAAAPQILCFLQSFWTGSYIDSNINVNDGRTGKDVNSIISSI
HTFDPASACTDATFQPCSSRALLNHKAVTDSFRTVYGINKGIAQGQAVAVG
RYSEDVYYNGNPWYLATLAAAEQLYAAVYQWNTIGSITVDSVSLPFFKDLL
PSIATGTYASSSATFASIVSAVKTYGDGYVAVVQKYTPANGGLAEQFDKSS
GSPLSAVDLTWSYAAFLTATDRRSGSVGPSWGEKSNNVPPTSCTAPPSCNV
QVTFNERVTTAYGDNIFIVGQLTQLGNWDPNSAVALSASKYTSSDPLWYAT
VSLPASTSFAYKYIKKTSSGTVVWESDPNRSYTTATTCGSTATQNDTWR

### Example 2

### Expression of Paraphaeosphaeria sporulosa Glucoamylase (PspGA3)

The nucleotide sequence of the PspGA3 gene from Paraphaeosphaeria sporulosa synthesized by Generay (Generay Biotech Co., Ltd, Shanghai, China) is set forth as SEQ ID NO. 4:

ATGCTGTTCACCAGCGTCATTCGAGCTCTCCCCGCGTCTCTCCTGTTCACC
GGCGCCCACGGCACCCCTTTGGAACGACGACAGACCAGCGTTGACACCTTC
GTTACCTCTCAGCGATCCGTTTCCATCCAGGGCGTTCTGTCTAACATTGGC
GCTGACGGCAGCAAGGCTCAGGGCGCTGCTGCTGGCATCGTCGTTGCTTCT
CCTAGCAAGTCCAACCCCGACTACTGGTACACCTGGACCCGAGACTCCGCC
CTGACCTACAAGGCTCTGGTTGAGCGATTCATTGGCGGCGACACCACCCTG
CGAAAGAAGCTGGACGAATACGTTAGCAGCCAGGCTTACCTGCAGACCGTT
TCTAACCCTTCCGGCGGCCCCGACACCGGCGGCCTGGGCGAGCCTAAGTTC
AACGTCGACCGAACCGCTTTCACCGGCGCTTGGGGCCGCCCTCAGCGAGAC
GGCCCTCCTCTCCGAGCTACCGCTCTGACCCTGTACGCCAACTGGCTGATT
GCTAACGGCAACACCACCCAGGCTGCCAACACCCTGTGGCCCGTCATTGCC
AAGGACCTCGCTTACGCCGTTAAGTACTGGAACCAGACCGGCTTCGACCTG
TGGGAAGAGGTTAACGGCTCTTCTTTCTTCACCCTGGCTGCTACCCACCGA
GCTCTCGTCGAGGGCGCCGCTCTCGCCACCAAGCTGTCCAAGACCTGCGAC
GGCTGCGCCGCTGCTGCTCCTCAGATTCTGTGCTTCCTGCAGTCTTTCTGG
ACCGGCTCTTACATCGACTCTAACATTAACGTCAACGACGGCCGAACCGGC
AAGGACGTCAACTCTATTATTTCTAGCATCCACACCTTCGACCCCGCCTCT
GCTTGCACCGACGCTACCTTCCAGCCTTGCAGCTCCCGAGCTCTCCTGAAC
CACAAGGCTGTTACCGACTCTTTCCGAACCGTTTACGGCATCAACAAGGGC
ATTGCTCAGGGCCAGGCTGTTGCTGTTGGCCGATACTCTGAGGACGTTTAC
TACAACGGCAACCCTTGGTATCTCGCTACCCTCGCTGCCGCTGAGCAGCTG
TACGCCGCCGTTTACCAGTGGAACACCATTGGCTCTATTACCGTTGACAGC
GTCAGCCTCCCTTTCTTCAAGGACCTCCTGCCTAGCATCGCCACCGGCACC
TACGCTTCTAGCAGCGCCACCTTCGCTTCCATTGTTAGCGCCGTCAAGACC
TACGGCGACGGCTACGTCGCCGTTGTTCAGAAGTACACCCCCGCCAACGGC
GGCCTCGCTGAGCAGTTCGACAAGTCTAGCGGCAGCCCTCTGTCTGCCGTT
GACCTGACCTGGTCTTACGCCGCTTTCCTCACCGCTACCGACCGACGATCT
GGCAGCGTTGGCCCTTCTTGGGGCGAGAAGTCTAACAACGTCCCTCCTACC
TCTTGCACCGCCCCTCCTTCTTGCAACGTCCAGGTTACCTTCAACGAGCGA
GTTACCACCGCCTACGGCGACAACATTTTCATTGTTGGCCAGCTCACCCAG
CTGGGCAACTGGGACCCTAACTCTGCCGTTGCCCTGTCTGCTTCTAAGTAC
ACCAGCTCTGACCCCCTGTGGTACGCTACCGTTAGCCTCCCCGCGTCCACC
AGCTTCGCCTACAAGTACATTAAGAAGACCAGCTCCGGCACCGTCGTTTGG
GAGTCTGACCCCAACCGATCTTACACCACCGCTACCACCTGCGGCAGCACC
GCTACCCAGAACGACACCTGGCGCTGA

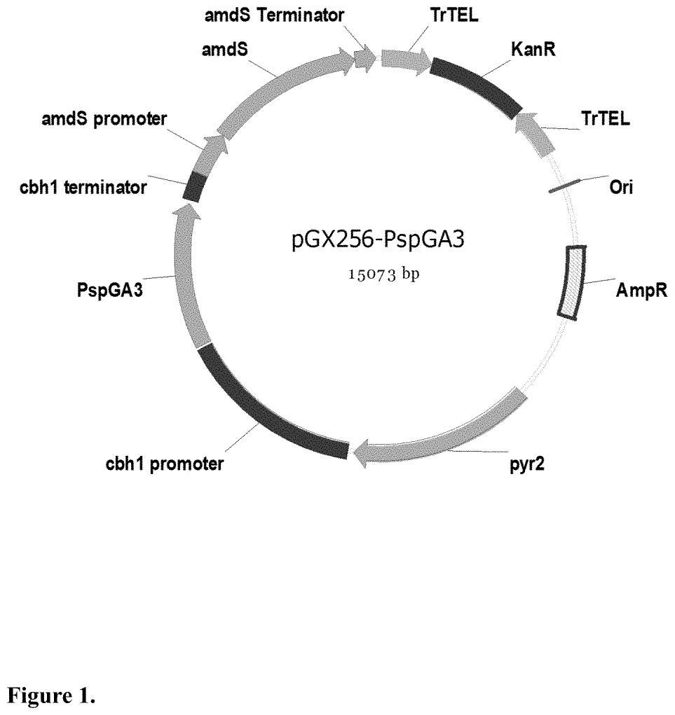

The DNA sequence of PspGA3 was optimized for expression of PspGA3 in Trichoderma reesei and inserted into the pGX256 expression vector (described in U.S. Published Application 2011/0136197 A1), resulting in pGX256-PspGA3 (FIG. 1).

The plasmid pGX256-PspGA3 was transformed into a suitable Trichoderma reesei strain (described in WO 05/001036) using protoplast transformation (Te'o et al., J. Microbiol. Methods 51:393-99, 2002). The transformants were selected and fermented by the methods described in WO 2016/138315. Supernatants from these cultures were used to confirm the protein expression by SDS-PAGE analysis and assay for enzyme activity.

A seed culture of the transformed cells mentioned, above, was subsequently grown in a 2.8 L fermenter in a defined medium. Fermentation broth was sampled at elapsed fermentation times of 42 hours, 65 hours, and 95 hours to run SDS-PAGE analysis, measurements of dry cell weight, residual glucose, and extracellular protein concentration. FIG. 2 displayed the production profile of PspGA3 within 95-hour fermentation. Following centrifugation, filtration and concentration, 500 mL of the concentrated sample was obtained. The protein quantification using the BCA method (protein quantification kit, Shanghai Generay Biotech CO., Ltd) demonstrated that the concentration of protein in the concentrated sample was 10.7 g/L.

### Example 3

### Purification of PspGA3

PspGA3 was purified via the beta-cyclodextrin coupled Sepharose 6 affinity chromatography, taking advantage of its carbohydrate binding domain. About 800 mL crude broth was received from shake flask. The solution was concentrated to about 80 mL, and then loaded onto a 50-mL beta-cyclodextrin coupled Sepharose 6 column (pre-equilibrated with 20 mM sodium acetate pH 5.0, 150 mM NaCl). After washing with the same buffer for 4 column volumes, the column was applied with 10 mM alpha-cyclodextrin in 20 mM sodium acetate pH 5.0 and 150 mM NaCl buffer for 5 column volumes. The fractions from the column were assayed for glucoamylase activity and SDS-PAGE. The fractions containing the target protein were pooled, concentrated and exchanged buffer to 20 mM sodium acetate pH 5.0, 150 mM NaCl using an Amicon Ultra-15 device with 10 K Amicon Ultra devices (Millipore). The purified sample is above 95% pure and stored in 40% glycerol at −80° C. until usage.

### Example 4

### Specific Activity of PspGA3 on Soluble Starch

Glucoamylase specific activity was assayed based on the release of glucose by glucoamylase from soluble starch. The rate of glucose release was measured using a coupled glucose oxidase/peroxidase (GOX/HRP) method (Anal. Biochem. 105 (1980), 389-397). Glucose was quantified as the rate of oxidation of 2,2′-Azino-bis 3-ethylbenzothiazoline-6-sulfonic acid (ABTS) by peroxide which was generated from coupled GOX/HRP enzymes reacted with glucose.

Substrate solutions were prepared by mixing 9 mL of soluble starch (1% in water, w/w) and 1 mL of 0.5 M pH 5.0 sodium acetate buffer in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX.

Serial dilutions of glucoamylase samples and glucose standard were prepared in Milli Q water. Each glucoamylase sample (10 μL) was transferred into a new microtiter plate (Corning 3641) containing 90 μL of substrate solution preincubated at 50° C. for 5 min at 600 rpm. The reactions were carried out at 50° C. for 10 min with shaking (600 rpm) in a thermomixer (Eppendorf), 10 μL of reaction mixtures as well as 10 μL of serial dilutions of glucose standard were quickly transferred to new microtiter plates (Corning 3641), respectively, followed by the addition of 100 μL of ABTS/GOX/HRP solution. The microtiter plates containing the reaction mixture were immediately measured at 405 nm at 11 seconds intervals for 5 min on SoftMax Pro plate reader (Molecular Device). The output was the reaction rate, Vo, for each enzyme concentration. Linear regression was used to determine the slope of the plot Vo vs. enzyme dose. The specific activity of glucoamylase was calculated based on the glucose standard curve using Equation 1:

Specific Activity (Unit/mg)=Slope (enzyme)/slope (std)×1000 (1),
where 1 Unit=1 μmol glucose/min.

Using the method mentioned above, specific activity of PspGA3 was determined and compared with the benchmarks, AnGA (a glucoamylase from Aspergillus niger, purified from DuPont product, Optidex L-400), FvGA (a glucoamylase from Fusarium verticilloides, referring to WO2016100871A1), AfuGA (described in WO2014092960) and TrGA (Trichoderma reesei glucoamylase, purified from DuPont product, STARGEN™ 002). Results are shown in Table 2. PspGA3 showed specific activity of 367 U/mg towards soluble starch, this results in approximately 2 fold higher activity compared to AnGA, 1.3 fold higher compared to FvGA and TrGA, and 1.6 fold higher compared to AfuGA.

TABLE 2
Specific activity of purified PspGA3 towards soluble
starch compared to other glucoamylases

| | Specific activity (U/mg/min) | | | | |
|---|---|---|---|---|---|
| Substrate | PspGA3 | AnGA | FvGA | AfuGA | TrGA |
| Soluble | 367.0 | 184.8 | 281.2 | 224.2 | 292.0 |
| Starch | | | | | |

### Example 5

### Pullulan-Hydrolyzing Activity of Glucoamylase PspGA3

Glucoamylase activity towards pullulan was assayed using the same protocol as described above for specific activity of glucoamylase PspGA3 towards soluble starch, except that the enzymes was dosed at 10 ppm. Table 3 summarizes pullulan-hydrolyzing activities of PspGA3 as well as the benchmarks. The pullulan hydrolyzing activity of PspGA3 was approximately 2.5 fold higher than that of AnGA, 2.1 fold higher than that of AfuGA and 1.8 fold higher than that of TrGA.

TABLE 3
Pullulan-hydrolyzing activity of PspGA3
compared with other glucoamylases

| | GA (dosed at 10 ppm) activity | | | | |
|---|---|---|---|---|---|
| | towards pullulan | | | | |
| Substrate | PspGA3 | AnGA | FvGA | AfuGA | TrGA |
| Pullulan | 124.2 | 50.0 | 207.7 | 58.5 | 68.4 |

### Example 6

### pH Effect and Temperature Effect on PspGA3 Glucoamylase Activity

The effect of pH (from 3.0 to 10.0) on PspGA3 activity was monitored using soluble starch (1% in water, w/w) as substrate. Buffer working solutions consisted of the combination of glycine/sodium acetate/HEPES (250 mM), with pH varying from 3.0 to 10.0. Substrate solutions were prepared by mixing soluble starch (1% in water, w/w) with 250 mM buffer solution at a ratio of 9:1. Enzyme working solutions were prepared in water at a certain dose (showing signal within linear range as per dose response curve). All the incubations were carried out at 50° C. for 10 min following the same protocol as described above for specific activity of glucoamylase PspGA3 towards soluble starch. Enzyme activity at each pH was reported as relative activity compared to enzyme activity at optimum pH. The pH profile of PspGA3 is shown in Table 4. PspGA3 was found to have an optimum pH at about 5.0 and retaining greater than 70% of maximum activity between pH 3.7 and 7.4.

TABLE 4
pH profile of PspGA3

| pH | Relative activity (%) |
|---|---|
| 3 | 38 |
| 4 | 83 |
| 5 | 100 |
| 6 | 99 |
| 7 | 87 |
| 8 | 48 |
| 9 | 24 |
| 10 | 20 |

The effect of temperature (from 40° C. to 90° C.) on PspGA3 activity was monitored using soluble starch (1% in water, w/w) as substrate. Substrate solutions were prepared by mixing 9 mL of soluble starch (1% in water, w/w) and 1 mL of 0.5 M buffer (pH 5.0 sodium acetate) into a 15-mL conical tube. Enzyme working solutions were prepared in water at a certain dose (showing signal within linear range as per dose response curve). Incubations were performed at temperatures from 40° C. to 90° C., respectively, for 10 min following the same protocol as described above for specific activity of glucoamylase PspGA3 towards soluble starch. Activity at each temperature was reported as relative activity compared to enzyme activity at optimum temperature. The temperature profile of PspGA3 is shown in Table 5. PspGA3 was found to have an optimum temperature of 63° C. and was able to keep higher than 70% of maximum activity between 52° C. and 69° C.

TABLE 5
Temperature-activity profile of PspGA3

| Temp. (° C.) | Relative activity (%) |
|---|---|
| 40 | 43 |
| 44.7 | 54 |
| 49.4 | 61 |
| 55 | 80 |
| 59.7 | 93 |
| 63 | 100 |
| 65 | 99 |
| 70 | 62 |
| 74.1 | 18 |
| 79.5 | 5 |
| 85.1 | 2 |
| 90 | 2 |

### Example 7

### Saccharification Evaluation of PspGA3 at pH 4.5, 60° C.

The goal of this study was to compare the saccharification performance of PspGA3 against the benchmarks, AnGA, FvGA TrGA and AfuGA under conventional saccharification conditions (pH 4.5, 60° C.). The evaluation of DP1 production of glucoamylase samples was performed by analyzing sugar compositions with equal enzyme dosage. Alpha-amylase-pretreated corn starch liquefact (34.9% ds, pH 4.68) was got from local Haocheng company and used as a starting substrate. The incubations of gluco-amylases (dosed at 25 or 50 μg/gds) and corn starch liquefact (32% ds) were performed at pH 4.5, 60° C. Samples were collected at 24, 48, and 72 h, respectively. All the incubations were quenched by heating at 100° C. for 15 min. Supernatant of the sample was transferred and diluted 400-fold in 5 mM H2SO4 for HPLC analysis. HPLC separation was performed using an Agilent 1200 series HPLC system with a Fast fruit column (100 mm×7.8 mm) at 80° C. The sample (10 μL) was subjected to the HPLC column and separated with an isocratic gradient of Milli-Q water as the mobile phase at a flow rate of 1.0 mL/min. The oligosaccharide products were detected using a refractive index detector. The glucogenic activities of the samples are summarized in Table 6. PspGA3 showed higher glucogenic activity on corn starch liquefact than AnGA, FvGA, TrGA, and AfuGA at both dosages of 25 μg/gds and 50 μg/gds. Especially when the enzyme was dosed at 50 μg/gds with 72 h incubation, PspGA3 could reach 94% of DP1 production, which is a big improvement with 3% more of DP1 release compared with AnGA at the same condition.

TABLE 6
Sugar composition analysis of PspGA3, AnGA, FvGA,
TrGA, and AfuGA incubated with corn starch liquefact
(32% ds) at pH 4.5, 60° C., respectively.

| | Enzyme | Time | | | | |
|---|---|---|---|---|---|---|
| Sample | Dose | (h) | DP3+% | DP3% | DP2% | DP1% |
| PspGA3 | 25 μg/gds | 24 | 18.9 | 3.2 | 11.6 | 66.3 |
| | | 48 | 8.9 | 1.7 | 2.9 | 86.4 |
| | | 72 | 5.3 | 1.4 | 2.3 | 91.0 |
| | 50 μg/gds | 24 | 10.6 | 1.5 | 4.1 | 83.8 |
| | | 48 | 4.0 | 1.1 | 2.7 | 92.2 |
| | | 72 | 2.1 | 0.8 | 3.1 | 94.0 |
| AnGA | 25 μg/gds | 24 | 23.9 | 5.2 | 19.0 | 51.9 |
| | | 48 | 15.4 | 1.7 | 7.3 | 75.5 |
| | | 72 | 10.4 | 2.0 | 2.4 | 85.2 |
| | 50 μg/gds | 24 | 17.4 | 2.1 | 10.3 | 70.3 |
| | | 48 | 8.6 | 1.7 | 2.1 | 87.7 |
| | | 72 | 5.7 | 1.4 | 2.0 | 90.9 |
| FvGA | 25 μg/gds | 24 | 28.8 | 8.7 | 13.2 | 49.4 |
| | | 48 | 11.4 | 1.4 | 7.4 | 79.7 |
| | | 72 | 7.2 | 1.6 | 3.7 | 87.6 |
| | 50 μg/gds | 24 | 9.5 | 1.4 | 4.6 | 84.5 |
| | | 48 | 3.7 | 1.2 | 2.6 | 92.5 |
| | | 72 | 2.5 | 1.0 | 2.8 | 93.8 |
| TrGA | 25 μg/gds | 24 | 16.4 | 1.7 | 9.7 | 72.1 |
| | | 48 | 10.6 | 1.5 | 3.3 | 84.6 |
| | | 72 | 7.7 | 1.1 | 2.9 | 88.3 |
| | 50 μg/gds | 24 | 11.9 | 1.3 | 4.0 | 82.7 |
| | | 48 | 6.6 | 0.9 | 3.4 | 89.1 |
| | | 72 | 4.4 | 0.8 | 4.1 | 90.7 |
| AfuGA | 25 μg/gds | 24 | 26.1 | 6.1 | 14.6 | 53.3 |
| | | 48 | 12.2 | 1.6 | 4.1 | 82.1 |
| | | 72 | 7.6 | 1.8 | 1.8 | 88.7 |
| | 50 μg/gds | 24 | 14.1 | 1.5 | 5.2 | 79.2 |
| | | 48 | 5.9 | 1.5 | 2.0 | 90.7 |
| | | 72 | 3.9 | 1.1 | 2.1 | 92.9 |

What is claimed is:

1. A method for saccharifying a starch substrate, comprising contacting the starch substrate with a glucoamylase selected from the group consisting of:
(a) a polypeptide having the amino acid sequence of SEQ ID NO: 3;
(b) a polypeptide having at least 81% identity to the amino acid sequence of SEQ ID NO: 3;
(c) a polypeptide having at least 82% identity to a catalytic domain of SEQ ID NO: 3; or
(d) a polypeptide having at least 82% identity to a linker and a catalytic domain of SEQ ID NO: 3.

2. The method of claim 1, wherein saccharifying the starch substrate results in a high glucose syrup.

3. The method of claim 1 or 2, wherein the high glucose syrup comprises an amount of glucose selected from the list consisting of at least 95.5% glucose, at least 95.6% glucose, at least 95.7% glucose, at least 95.8% glucose, at least 95.9% glucose, at least 96% glucose, at least 96.1% glucose, at least 96.2% glucose, at least 96.3% glucose, at least 96.4% glucose, at least 96.5% glucose and at least 97% glucose.

4. The method of any one of claims 1-3, further comprising fermenting the high glucose syrup to an end product.

5. The method of claim 4, wherein saccharifying and fermenting are carried out as a simultaneous saccharification and fermentation (SSF) process.

6. The method of claim 4 or claim 5, wherein the end product is alcohol, for example, ethanol.

7. The method of claim 4 or claim 5, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.

8. The method of any one of claims 1-7, wherein the starch substrate is about, 5% to 99%, 15% to 50% or 40-99% dry solid (DS).

9. The method of any one of claims 1-8, wherein the starch substrate is selected from wheat, barley, corn, rye, rice, sorghum, bran, cassava, milo, millet, potato, sweet potato, tapioca, and any combination thereof.

10. The method of any one of claims 1-9, wherein the starch substrate comprises liquefied starch, gelatinized starch, or granular starch.

11. The method of any one of claims 1-10, further comprising adding a hexokinase, a xylanase, a glucose isomerase, a xylose isomerase, a phosphatase, a phytase, a pullulanase, a beta-amylase, an a-amylase, a protease, a cellulase, a hemicellulase, a lipase, a cutinase, a trehalase, an isoamylase, a redox enzyme, an esterase, a transferase, a pectinase, a hydrolase, an alpha-glucosidase, a beta-glucosidase, or a combination thereof to the starch substrate.

12. A method for saccharifying and fermenting a starch substrate to produce an end product, comprising contacting the starch substrate with a glucoamylase selected from the group consisting of:
a) a polypeptide having the amino acid sequence of SEQ ID NO: 3;
b) a polypeptide having at least 81% identity to the amino acid sequence of SEQ ID NO: 3;
c) a polypeptide having at least 82% identity to a catalytic domain of SEQ ID NO: 3; or
d) a polypeptide having at least 82% identity to a linker and a catalytic domain of SEQ ID NO: 3.

13. The method of claim 12, wherein saccharifying and fermenting are carried out as a simultaneous saccharification and fermentation (SSF) process.

14. The method of claim 12 or claim 13, wherein the end product is alcohol, for example, ethanol.

15. The method of claim 12, wherein the saccharified starch substrate results in a reduced level of DP3+ and an increased level of DP1 compared to contacting the same starch substrate with AnGA.

16. The method of claim 12 or claim 13, wherein the end product is a biochemical selected from the group consisting of an amino acid, an organic acid, citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, lysine, itaconic acid, 1,3-propanediol, biodiesel, and isoprene.

17. A method of producing a fermented beverage, wherein the method comprises the step of contacting a mash and/or a wort with a glucoamylase selected from the group consisting of:
a) a polypeptide having the amino acid sequence of SEQ ID NO: 3;
b) a polypeptide having at least 81% identity to the amino acid sequence of SEQ ID NO: 3;
c) a polypeptide having at least 82% identity to a catalytic domain of SEQ ID NO: 3; or
d) a polypeptide having at least 82% identity to a linker and a catalytic domain of SEQ ID NO: 3.


SEQUENCE LISTING (4 sequences)

SEQ ID NO: 1 - DNA, 1810 bp, Paraphaeosphaeria sporulosa
atgctcttcacctcagtcattcgggcccttccggcatccttgctttttacaggcgctcatggcacgcctctcgagcgtcgtcagacttccgtcgacacgttcgtaacaagccagagatcggtttcgatccaaggtgtgctttctaacatcggggctgatggctccaaggcgcaaggtgcagcagcgggcatcgtcgtggccagtccgtccaagtcaaacccagattgtaagtagaacaaactcaggcgcacattacatgggttgacaagccatagactggtacacttggacaagagactctgctttgacgtacaaagccctcgtcgagcgtttcatcggtggggacaccacactgagaaagaagctcgatgagtacgtctcatcccaggcgtatctccagaccgtgtcgaacccgtccggcggtcctgacaccggaggactcggcgagcccaagttcaacgtcgaccgtactgcatttactggagcttggggtcgtcctcagcgcgatggaccacctctacgagccactgccttgacactctacgctaattggcttattgccaacggcaacaccacacaagctgcaaataccctttggcctgttattgccaaggatcttgcctacgccgtcaagtactggaaccagaccggcttcgacttgtgggaagaggtcaatggctcatccttctttaccctcgcagctactcatcgtgccctcgtagagggcgctgcactcgctactaagctctcaaagacatgtgacgggtgtgctgccgcggccccacagatcctctgcttccttcaaagcttctggactggcagttacatcgattccaacatcaatgtgaatgatggtcgtactggcaaagacgtcaactccatcatctcatcgatccataccttcgacccagcgtcagcctgtaccgacgctactttccaaccttgctcttctcgcgctctgctcaaccacaaggctgttactgactccttccgcaccgtgtacggcatcaacaagggtatcgcccaaggacaagctgtcgccgttggaagatattctgaggatgtatactacaatggcaacccgtggtatcttgcgacgctcgcggccgcagaacaactttacgccgctgtctaccaatggaacaccataggctccatcaccgttgactctgtatctcttccattcttcaaggacctcctgcccagcatcgccactggtacctacgcctcgagctcggctaccttcgcatcgattgtctctgctgtcaaaacatacggtgacggctacgttgccgtggtccagaagtacactcctgccaatggcgggttggccgaacagttcgacaagagcagtggttccccactctctgccgttgacctgacttggtcttatgcggctttcctgaccgccaccgatcgtcgttctgggtctgtgggtccttcttggggagagaagtccaacaacgttcctccgacatcatgcacagctcctccttcctgcaatgtgcaagtcactttcaacgaacgcgtgaccaccgcctacggagacaacatcttcattgttggacagctcactcagctcggcaactgggatccgaacagcgctgttgcgctcagtgccagcaagtacaccagcagtgacccgctctggtatgcaactgtcagcctccctgcctcaacttctttcgcatacaaatacatcaagaagacgtcgagcggcactgtcgtatgggagagcgacccgaacaggagctacacaactgcaaccacttgtggaagcactgcgactcagaacgacacatggagataa

SEQ ID NO: 2 - protein, 586 aa, Paraphaeosphaeria sporulosa
MLFTSVIRALPASLLFTGAHGTPLERRQTSVDTFVTSQRSVSIQGVLSNIGADGSKAQGAAAGIVVASPSKSNPDYWYTWTRDSALTYKALVERFIGGDTTLRKKLDEYVSSQAYLQTVSNPSGGPDTGGLGEPKFNVDRTAFTGAWGRPQRDGPPLRATALTLYANWLIANGNTTQAANTLWPVIAKDLAYAVKYWNQTGFDLWEEVNGSSFFTLAATHRALVEGAALATKLSKTCDGCAAAAPQILCFLQSFWTGSYIDSNINVNDGRTGKDVNSIISSIHTFDPASACTDATFQPCSSRALLNHKAVTDSFRTVYGINKGIAQGQAVAVGRYSEDVYYNGNPWYLATLAAAEQLYAAVYQWNTIGSITVDSVSLPFFKDLLPSIATGTYASSSATFASIVSAVKTYGDGYVAVVQKYTPANGGLAEQFDKSSGSPLSAVDLTWSYAAFLTATDRRSGSVGPSWGEKSNNVPPTSCTAPPSCNVQVTFNERVTTAYGDNIFIVGQLTQLGNWDPNSAVALSASKYTSSDPLWYATVSLPASTSFAYKYIKKTSSGTVVWESDPNRSYTTATTCGSTATQNDTWR

SEQ ID NO: 3 - protein, 559 aa, Paraphaeosphaeria sporulosa
QTSVDTFVTSQRSVSIQGVLSNIGADGSKAQGAAAGIVVASPSKSNPDYWYTWTRDSALTYKALVERFIGGDTTLRKKLDEYVSSQAYLQTVSNPSGGPDTGGLGEPKFNVDRTAFTGAWGRPQRDGPPLRATALTLYANWLIANGNTTQAANTLWPVIAKDLAYAVKYWNQTGFDLWEEVNGSSFFTLAATHRALVEGAALATKLSKTCDGCAAAAPQILCFLQSFWTGSYIDSNINVNDGRTGKDVNSIISSIHTFDPASACTDATFQPCSSRALLNHKAVTDSFRTVYGINKGIAQGQAVAVGRYSEDVYYNGNPWYLATLAAAEQLYAAVYQWNTIGSITVDSVSLPFFKDLLPSIATGTYASSSATFASIVSAVKTYGDGYVAVVQKYTPANGGLAEQFDKSSGSPLSAVDLTWSYAAFLTATDRRSGSVGPSWGEKSNNVPPTSCTAPPSCNVQVTFNERVTTAYGDNIFIVGQLTQLGNWDPNSAVALSASKYTSSDPLWYATVSLPASTSFAYKYIKKTSSGTVVWESDPNRSYTTATTCGSTATQNDTWR

SEQ ID NO: 4 - DNA, 1761 bp, Paraphaeosphaeria sporulosa
atgctgttcaccagcgtcattcgagctctccccgcgtctctcctgttcaccggcgcccacggcacccctttggaacgacgacagaccagcgttgacaccttcgttacctctcagcgatccgtttccatccagggcgttctgtctaacattggcgctgacggcagcaaggctcagggcgctgctgctggcatcgtcgttgcttctcctagcaagtccaaccccgactactggtacacctggacccgagactccgccctgacctacaaggctctggttgagcgattcattggcggcgacaccaccctgcgaaagaagctggacgaatacgttagcagccaggcttacctgcagaccgtttctaacccttccggcggccccgacaccggcggcctgggcgagcctaagttcaacgtcgaccgaaccgctttcaccggcgcttggggccgccctcagcgagacggccctcctctccgagctaccgctctgaccctgtacgccaactggctgattgctaacggcaacaccacccaggctgccaacaccctgtggcccgtcattgccaaggacctcgcttacgccgttaagtactggaaccagaccggcttcgacctgtgggaagaggttaacggctcttctttcttcaccctggctgctacccaccgagctctcgtcgagggcgccgctctcgccaccaagctgtccaagacctgcgacggctgcgccgctgctgctcctcagattctgtgcttcctgcagtctttctggaccggctcttacatcgactctaacattaacgtcaacgacggccgaaccggcaaggacgtcaactctattatttctagcatccacaccttcgaccccgcctctgcttgcaccgacgctaccttccagccttgcagctcccgagctctcctgaaccacaaggctgttaccgactctttccgaaccgtttacggcatcaacaagggcattgctcagggccaggctgttgctgttggccgatactctgaggacgtttactacaacggcaacccttggtatctcgctaccctcgctgccgctgagcagctgtacgccgccgtttaccagtggaacaccattggctctattaccgttgacagcgtcagcctccctttcttcaaggacctcctgcctagcatcgccaccggcacctacgcttctagcagcgccaccttcgcttccattgttagcgccgtcaagacctacggcgacggctacgtcgccgttgttcagaagtacacccccgccaacggcggcctcgctgagcagttcgacaagtctagcggcagccctctgtctgccgttgacctgacctggtcttacgccgctttcctcaccgctaccgaccgacgatctggcagcgttggcccttcttggggcgagaagtctaacaacgtccctcctacctcttgcaccgcccctccttcttgcaacgtccaggttaccttcaacgagcgagttaccaccgcctacggcgacaacattttcattgttggccagctcacccagctgggcaactgggaccctaactctgccgttgccctgtctgcttctaagtacaccagctctgaccccctgtggtacgctaccgttagcctccccgcgtccaccagcttcgcctacaagtacattaagaagaccagctccggcaccgtcgtttgggagtctgaccccaaccgatcttacaccaccgctaccacctgcggcagcaccgctacccagaacgacacctggcgctga